(19) United States
(12) Patent Application Publication
(10) Pub. No.: US 2024/0216594 A1
(43) Pub. Date: Jul. 4, 2024

(54) DIALYSIS SOLUTION BAG ADAPTOR SYSTEM

(71) Applicant: Fresenius Medical Care Holdings, Inc., Waltham, MA (US)

(72) Inventors: David Yuds, Hudson, NH (US); Richard Allen Lasher, Knoxville, TN (US)

(21) Appl. No.: 18/149,978

(22) Filed: Jan. 4, 2023

(51) Int. Cl. A61M 1/16; A61M 1/26; A61M 1/28; A61M 39/28

(52) U.S. Cl. CPC A61M 1/1668; A61M 1/267; A61M 1/282; A61M 39/28; A61M 2039/1077; A61M 2205/3375

(57) ABSTRACT

Systems and methods are described for adapting dialysis solution bags between different modalities of dialysis treatment. Specifically, for example, the system described herein enables an automated peritoneal dialysis (APD) patient to use APD bags from their existing stock for a manual exchange treatment, such as a continuous ambulatory peritoneal dialysis (CAPD) treatment, thereby providing that the APD patient no longer needs to retain manual exchange bags and sets in the event of an emergency or other situation requiring the performing of manual dialysis exchanges.

### TECHNICAL FIELD

This application relates to a dialysis solution bag adaptor system, particularly in connection with adapting use of solution bags and disposable sets between different modalities of dialysis treatment.

### BACKGROUND

Medical devices, such as dialysis machines, are known for use in the treatment of renal disease. The two principal dialysis methods are hemodialysis (HD) and peritoneal dialysis (PD). During hemodialysis, the patient's blood is passed through a dialyzer of a hemodialysis machine while also passing dialysate through the dialyzer. A semi-permeable membrane in the dialyzer separates the blood from the dialysate within the dialyzer and allows diffusion and osmosis exchanges to take place between the dialysate and the blood stream. During peritoneal dialysis, the patient's peritoneal cavity is periodically infused with dialysate, or dialysis solution. The membranous lining of the patient's peritoneum acts as a natural semi-permeable membrane that allows diffusion and osmosis exchanges to take place between the solution and the blood stream. Different modalities of PD treatment include Continuous Ambulatory Peritoneal Dialysis (CAPD) and Automated Peritoneal Dialysis (APD). CAPD involves manually controlled fluid exchanges in which dialysis solution bags are hung from an IV pole with gravity used for flowing fluid into a patient's abdomen and then drained. A CAPD system uses an integrated tubing set that links the solution bag directly to a drain bag and requires no electricity to operate. In APD, machines called cyclers are designed to control the entire peritoneal dialysis process (fill, dwell and drain phases) so that it can be performed at home, usually overnight while a patient is sleeping, by automated operation. Both HD and PD machines may include displays with touch screens or other user interfaces that display information of a dialysis treatment and/or enable an operator or patient to interact with the machine.

PD patients who perform APD with a cycler use dialysis solution bags designed to connect to APD cycler sets. These bags come in a variety of sizes (e.g. 2 L, 3 L, 5 L, and 6 L) and glucose concentrations (e.g. 1.5%, 2.5%, and 4.25%). PD patients who perform manual peritoneal dialysis, CAPD, use a different set of dialysis solution bags as part of a CAPD system. These bags also come in a variety of sizes (2 L, 2.5 L, and 3 L) and glucose concentrations (1.5%, 2.5%, and 4.25%). CAPD sets feature a dialysis solution bag and a drain bag with a form of y-connection tubing to facilitate connecting a patient's PD catheter alternatingly to the fresh dialysis solution bag and then to the drain bag for spent dialysis solution. Many PD patients are initially trained on how to perform manual dialysis using CAPD sets regardless of whether or not they will use an APD cycler. This provides for the patient to learn to make safe aseptic connections and has the added benefit of providing an APD patient with the option for, and training on, performing manual dialysis as a back-up method for performing dialysis in the event of a power failure or other inability of an APD device to operate.

APD patients typically store a month's worth of APD supplies in their home at a time. Because the APD cycler uses different bags from manual CAPD bags, in order to have appropriate supplies for the manual back-up method of performing CAPD, the patient must also a minimum of a week's worth of CAPD supplies of a variety of glucose concentrations. This requires more storage space, frustrating patients who have limited room available. Furthermore, the expiration dates and inventory must be managed by the patient, rotating stock and disposing of expired solution bags—the patient may retain dozens of boxes of brand new CAPD sets for the entire span of its shelf life and, if no power-failure or other reason to perform CAPD has occurred during that time, the CAPD bags have to be cut open, drained, and discarded. This is costly, wasteful, and disposal may also be frustrating, especially when a patient has limited trash collection services.

Accordingly, it would be desirable to provide a system and method that addresses the above-noted concerns and other issues.

### SUMMARY

According to the system described herein, a system for adapting use of dialysis solution bags between modalities of dialysis treatment includes an adaptor assembly coupling a dialysis solution bag for a first modality of dialysis treatment to a disposable set configured for connection to a patient for a second modality of dialysis treatment. Fluid flow from the dialysis solution bag is controlled by the adaptor assembly for use with the second modality of dialysis treatment.

According further to the system described herein, a method for adapting use of a dialysis solution bag between modalities of dialysis treatment is provided. The method includes providing a dialysis solution bag for a first modality of dialysis treatment, and coupling the dialysis solution bag to a disposable set for a second modality of dialysis treatment via an adaptor assembly. The method further includes controlling flow of fluid from the dialysis solution bag via the adaptor assembly in a manner suitable for use with the second modality of dialysis treatment.

In various examples and implementations of the systems and methods described herein, the first modality of dialysis treatment may be an automated peritoneal dialysis (APD) and the dialysis solution bag may be an APD solution bag, and the second modality of dialysis treatment may be a manual exchange treatment and the disposable set may be a manual exchange disposable set. The adaptor assembly may include a manual exchange solution bag acting as a reservoir fill bag and be coupled to the APD solution bag via a connection adaptor device. The adaptor assembly may include a flow limiter device that limits fluid flow to the manual exchange disposable set. The flow limiter device may include an impeller that delivers a controlled amount of fluid to the manual exchange disposable set. The flow limiter device may include a clamping mechanism that engages to stop fluid flow, and the clamping mechanism may include a spring-loaded clamping mechanism. The flow limiter device may include an ultrasonic sensor that uses ultrasonic waves to determine fluid flow and that engages to limit fluid flow, and the ultrasonic sensor may trigger a clamping mechanism to stop fluid flow. The ultrasonic sensor may engage an ultrasonic piezo motor to trigger the clamping mechanism.

### BRIEF DESCRIPTION OF THE DRAWINGS

Implementations and features of the system described herein are explained with reference to the several figures of the drawings, which are briefly described as follows.

FIG. 1 shows a PD system including an automated peritoneal dialysis (APD) machine or cycler.

FIGS. 2A-2C depict a continuous ambulatory peritoneal dialysis (CAPD) treatment system as an example of a type of manual exchange system with which the system described herein can be practiced.

FIG. 3 is a schematic illustration of a device according to an implementation of the system described herein that adapts APD bags to perform CAPD.

FIG. 4 is a schematic illustration of another device according to another implementation of the system described herein in which the device may include a set of bags that allows fresh dialysate solution from an APD bag to fill a measured reservoir before being emptied into a patient.

FIGS. 5A and 5B are schematic illustrations of another implementation of the system described herein that allows the patient to connect to larger APD bag sizes using a durable or disposable flow limiter device that limits the flow to a smaller (e.g., 2 L or 3 L) fill volume.

FIG. 6 is a schematic illustration of an implementation of a device according to the system described that is battery powered and utilizing ultrasonics.

### DETAILED DESCRIPTION

According to the present disclosure, systems and methods are described for adapting dialysis solution bags between different modalities of peritoneal dialysis treatment. Specifically, for example, the system described herein enables an APD patient to use APD bags from their existing stock for a manual exchange treatment, such as a CAPD treatment, thereby providing that the APD patient no longer needs to retain manual exchange bags and sets in the event of an emergency or other situation requiring the performing of manual dialysis.

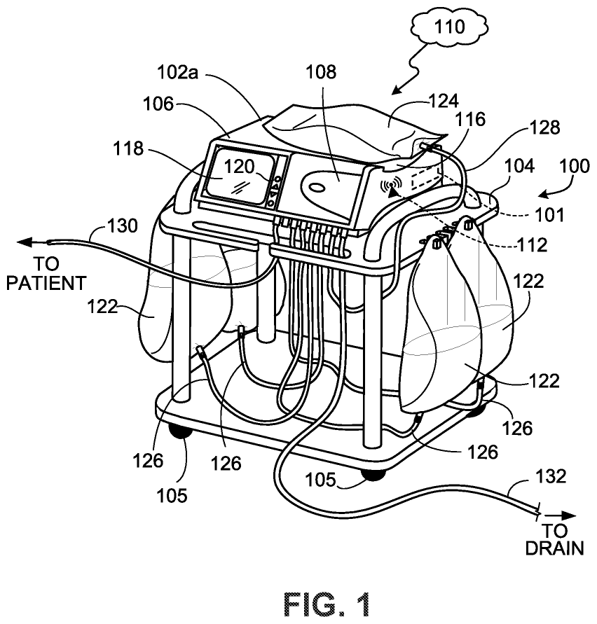

FIG. 1 is a schematic illustration of a PD system 100 including a dialysis machine 102, such as an APD machine or cycler. In some implementations, the dialysis machine 102 may be seated on a cabinet or cart 104 having one or more wheels 105 and be movable. The dialysis machine 102 may include a housing 106, a door 108, and a cartridge interface for contacting a disposable PD cassette, or cartridge, when the cartridge is disposed within a compartment formed between the cartridge interface and the closed door 108. A heater tray 116 may be positioned on top 102a of the housing 106. The heater tray 116 may be any size and shape to accommodate a bag of dialysate or dialysis solution (terms used interchangeably herein) (e.g., a 5 L bag of dialysate). It is noted that although the term “bag” is used herein, the system described herein may be used in connection with other types and forms of containers of dialysate. The dialysis machine 102 may also include a user interface such as a touch screen 118 and control panel 120 operable by a user (e.g., a caregiver or a patient) to allow, for example, set up, initiation, and/or termination of a PD treatment. A processing module 101 may be configured to receive data from the touch screen 118 the control panel 120 and sensors, e.g., temperature and pressure sensors, and control the dialysis machine 102 based on the received data. The dialysis machine 102 may include a connection component 112 configured to connect to a network 110 via a wired and/or wireless connection, such as via WiFi or Bluetooth.

Dialysate bags 122 may be suspended from the sides of the cart 104, and a heater bag 124 may be positioned in the heater tray 116. Hanging the dialysate bags 122 may improve air management as any air is disposed by gravity to a top portion of the dialysate bag 122. Valves may be attached to a bottom portion of the dialysate bags 122 so fluid is drawn out and air delivery is minimized. Dialysate from the dialysate bags 122 may be transferred to the heater bag 124 in batches. For example, a batch of dialysate may be transferred from the dialysate bags 122 to the heater bag 124, where the dialysate is heated by the heating element. When the batch of dialysate has reached a predetermined temperature (e.g., approximately 98°-100° F., 37° C.), the batch of dialysate may be flowed into the patient. The dialysate bags 122 and the heater bag 124 may be connected to the cartridge via dialysate bag lines 126 and a heater bag line 128, respectively. The dialysate bag lines 126 may be used to pass dialysate from dialysate bags 122 to the cartridge during use, and the heater bag line 128 may be used to pass dialysate back and forth between the cartridge and the heater bag 124 during use. In addition, a patient line 130 and a drain line 132 may be connected to the cartridge. The patient line 130 may be connected to a patient's abdomen via a catheter and may be used to pass dialysate back and forth between the cartridge and the patient's peritoneal cavity during use. The drain line 132 may be connected to a drain or drain receptacle and may be used to pass dialysate from the cartridge to the drain or drain receptacle during use.

As described herein, many PD patients are initially trained on how to perform manual dialysis using CAPD sets regardless of whether or not they will use an APD cycler. This provides for the patient to learn to make safe aseptic connections and has the added benefit of providing an APD patient with the option for, and training on, performing manual dialysis as a back-up method for performing dialysis in the event of a power failure or other inability of an APD device to operate. APD patients typically store a month's worth of APD supplies in their home at a time. Because the APD cycler uses different bags from manual CAPD bags, in order to have appropriate supplies for the manual back-up method of performing CAPD, the patient typically must also retain a minimum of a week's worth of CAPD supplies of a variety of glucose concentrations. It is noted that although CAPD is principally discussed herein as an applicable manual exchange treatment, the system described herein may be generally applied to any appropriate type of manual exchange system.

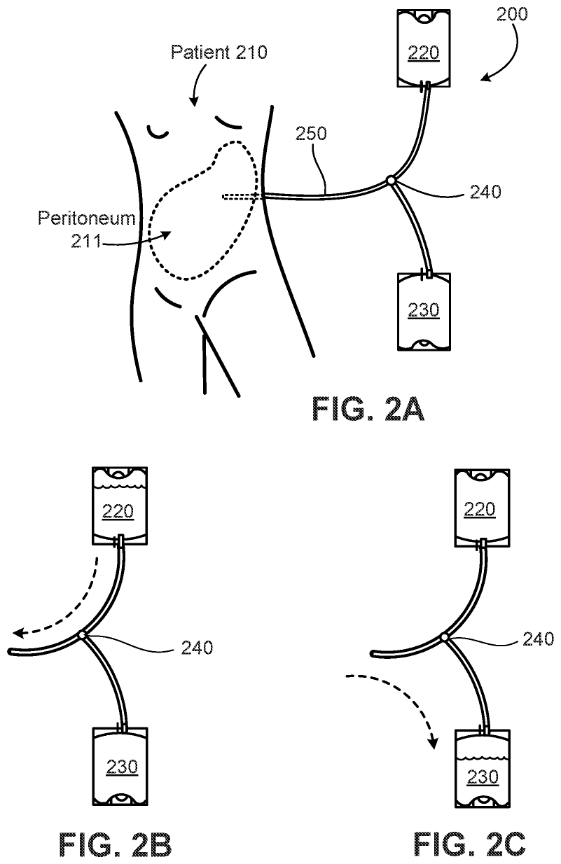

FIGS. 2A-2C depict a CAPD treatment system as an example of a type of manual exchange system according to which the system described herein can be practiced. FIG. 2A shows a CAPD treatment system 200 that includes a fresh PD solution supply bag 220, a spent PD solution bag 230, and a y-connector 240 for coupling those bags to peritoneal transfer set 250. Thus, for example, the patient connects bags 220, 230 to the y-connector 240, as shown in FIG. 2B, for a brief sterilizing flush of the connector 240. Then, as further shown in that drawing, the patient 210 configures the connector 240 to permit fresh PD solution to flow, under gravity assist, from bag 220 into the peritoneum 211. Once the PD solution has dwelled for the desired period, the patient 210 reconfigures the connector 240 to permit the spent PD solution to drain to bag 230 for disposal, as shown in FIG. 2C. The methods and systems described herein enable an APD patient to use APD bags from their existing stock for CAPD treatment, thereby providing hat the APD patient no longer needs to retain CAPD bags and sets in the event of an emergency or other situation requiring the performing of manual dialysis.

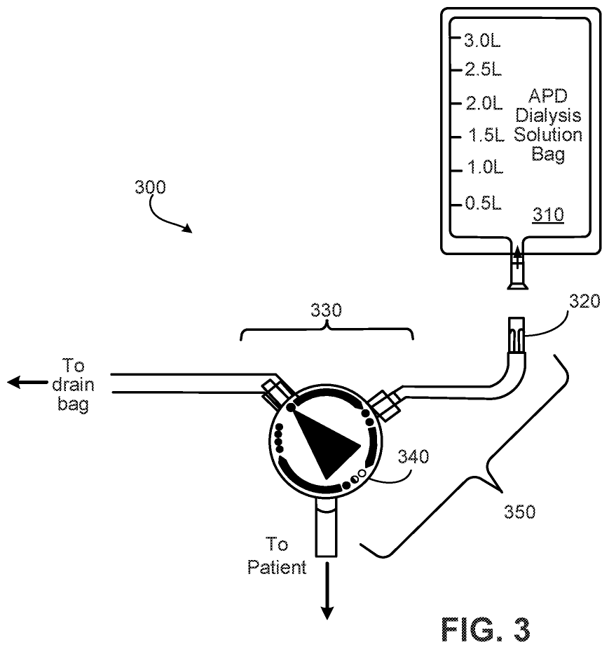

FIG. 3 is a schematic illustration of a system 300 having an adaptor device 350 according to an implementation of the system described herein that adapts APD bags to perform CAPD. As illustrated for the system 300, the adaptor device 350 may include a disposable y-shaped tubing set 330 with an APD bag adaptor 320 that connects the patient to an APD bag 310 and a drain bag to match the functions of a CAPD set, e.g., a 2 L or 3 L CAPD set. The APD bag adaptor 320 may include a male safe-lock connector. The implementation may optionally include a stay-safe disc 340, manufactured by Fresenius Medical Care, with features that guide a patient through a CAPD exchange, including “Fill” and “Drain” settings. Instructions for use would warn the patient to not connect APD bags larger than 2 or 3 L; however, since the same convention of the patient stopping flow when he or she feels full already exists, it would not be unacceptable to connect to larger bags and require the trained patient to stop the flow when the usual prescribed volume has been delivered. Keeping track of the delivered volume may be as simple as indicating the level on the side of the APD bag 310. This may be implemented using a sticker or other measuring meter applied to the side of the vertically hung bag 310. The described implementation of the system described herein provides a distinct advantage over filled CAPD bags for their shelf-life: non-fluid disposables can remain sterile for about 2.5 years whereas fluid filled CAPD bags have a shelf-life of about 18 months.

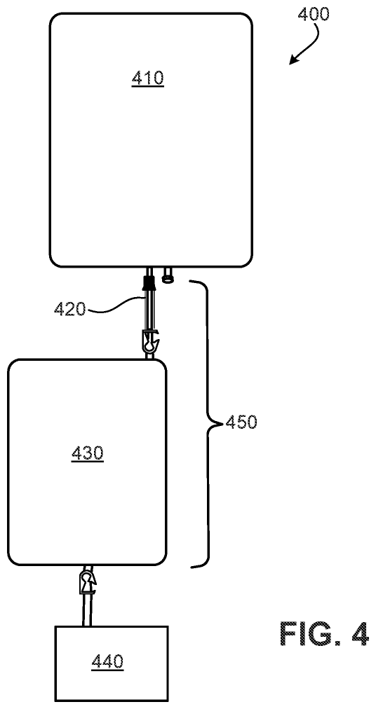

FIG. 4 is a schematic illustration of a system 400 including a device 450 according to another implementation of the system described herein for adapting APD bags to perform CAPD. As illustrated, the device 450 provides for use of a set of bags that allows fresh dialysate solution from an APD bag 410 (e.g. a 6 L APD dialysis solution bag) to fill a measured reservoir 430 before being emptied into a patient, e.g. the reservoir 430 may be 2 L CAPD dialysis solution fill reservoir. The device 450 includes an APD bag adaptor 420 to connect tubing of the measured reservoir 430 to the APD bag 410. The APD bag adaptor 420 may include an APD adaptor stalk. The device 450 may then provide for supply of dialysis solution to components of a regular CAPD system 440.

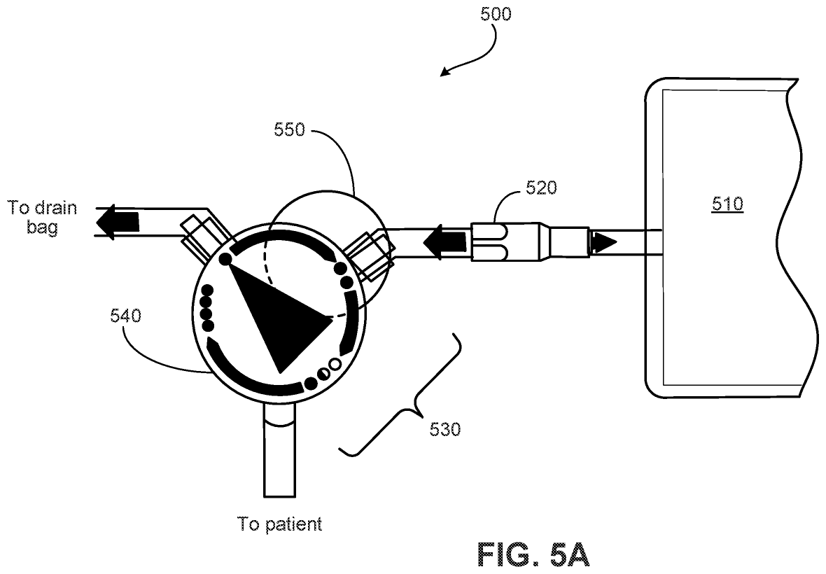

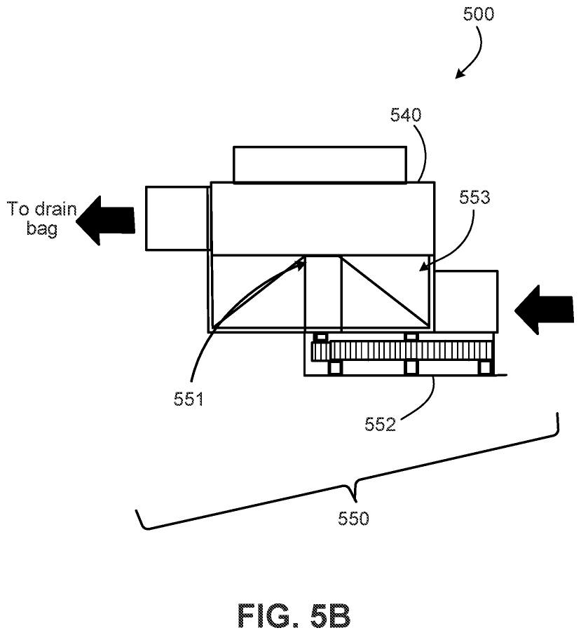

FIGS. 5A and 5B are schematic illustrations of another implementation of the system described herein including a durable or disposable flow limiter device 550. As shown in FIG. 5A, the system described herein allows a patient using a CAPD system 500 to connect, via connector 520, to a larger size APD bag 510 using the flow limiter device 550 that limits the flow to a smaller (e.g., 2 L or 3 L) fill volume. The device 550 may be coupled to a y-shaped tubing set 530 that may optionally include a stay-safe disk 540 like that manufactured by Fresenius Medical Care. Because the device 550 needs to run without a continuous source of electricity (i.e. during a power failure), the mechanism used to limit the flow could be either battery powered or mechanical, like a spring driven timer, storing potential energy.

As shown in FIG. 5B, the flow may be controlled by the device 550 using mechanical components that limit flow volumetrically and/or based on time or pressure. For example, as illustrated, the device 550 may include a mechanism 551 that is then engaged by a user, for example, when the stay-safe disk 540 is actuated to the “Fill” setting. Further, the device 550 may be include a gear assembly 552, e.g. including large and small gears, pegs, stops, with rotation based on flow that controls action of an impeller 553 that delivers a specified volume of fluid for every rotation. In other implementations, the flow may be limited either with a mechanical clamp operated by the patient or the device itself. In other implementations, the device 550 may produce a sound or turn on a light to indicate when the prescribed fill volume has been delivered to alert the patient.

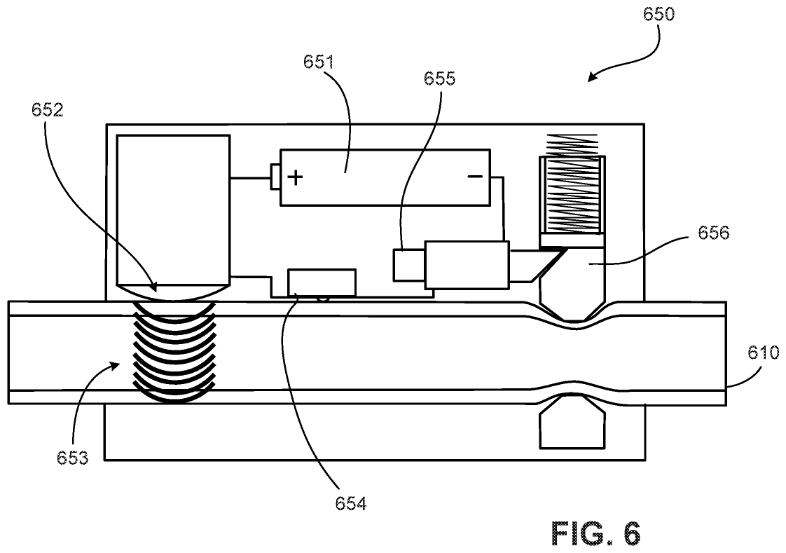

FIG. 6 is a schematic illustration of an implementation of a device 650 according to the system described that is battery powered and utilizing ultrasonics. The device 650 includes a battery 651. An ultrasonic sensor 652, using emission of ultrasonic waves 653, enables detection of flow of fresh dialysis fluid. A switch 654 engages the ultrasonic sensor 652 when tubing line 610 has been inserted. The empty tubing line 610 is used to automatically calibrate the sensor 651 before fluid flows through the tubing line 610. A motor 655 is used to prevent a spring-loaded clamping mechanism 656 from closing until the prescribed volume has been delivered. When triggered by a signal from the ultrasonic sensor 652, the motor causes the spring-loaded clamping mechanism 656 to engage and stop the flow of fluid. In an implementation, the motor 655 may be an ultrasonic piezo motor, such as a SQUIGGLE motor from New Scale Technologies. The motor 655 and spring mechanism 656 may be reset when opening a door that covers the device 650.

It is noted that CAPD bags are normally pre-connected to tubing to reduce the risk of peritonitis from touch contamination of bag connections. However, standard APD treatments rely on the patient making those critical connections to bags. This means that risk of peritonitis is no higher for the system described herein than that of a standard APD treatment. However, the risk of peritonitis can be further reduced with making those connections to APD bags incorporating appropriate sterilization measures, including use of peripheral devices and/or UV light sterilization.

Implementations discussed herein may be combined with each other in appropriate combinations in connection with the system described herein. Additionally, in some instances, the order of steps in the flow diagrams, flowcharts and/or described flow processing may be modified, where appropriate. The system may further include a display and/or other computer components for providing a suitable interface with a user and/or with other computers. Aspects of the system described herein may be implemented or controlled using software, hardware, a combination of software and hardware and/or other computer-implemented or computer-controlled modules or devices having described features and performing described functions. Data exchange and/or signal transmissions to, from and between components of the system may be performed using wired or wireless communication. This communication may include use of one or more transmitter or receiver components that securely exchange information via a network, such as the Internet, and may include use of components of local area networks (LANs) or other smaller scale networks, such as Wi-Fi, Bluetooth or other short range transmission protocols, and/or components of wide area networks (WANs) or other larger scale networks, such as mobile telecommunication networks.

Software implementations of aspects of the system described herein may include executable code that is stored in a computer-readable medium and executed by one or more processors. The computer-readable medium may include volatile memory and/or non-volatile memory, and may include, for example, a computer hard drive, ROM, RAM, flash memory, portable computer storage media, an SD card, a flash drive or other drive with, for example, a universal serial bus (USB) interface, and/or any other appropriate tangible or non-transitory computer-readable medium or computer memory on which executable code may be stored and executed by a processor. The system described herein may be used in connection with any appropriate operating system. The meanings of any method steps of the invention(s) described herein are intended to include any suitable method of causing one or more parties or entities to perform the steps unless a different meaning is expressly provided or otherwise clear from the context.

As used herein, an element or operation recited in the singular and preceded with the word “a” or “an” should be understood as not excluding plural elements or operations, unless such exclusion is explicitly recited. References to “one” embodiment or implementation of the present disclosure are not intended to be interpreted as excluding the existence of additional embodiments that also incorporate the recited features. Furthermore, a description or recitation in the general form of “at least one of [a], [b] or [c],” or equivalent thereof, should be generally construed to include [a] alone, [b] alone, [c] alone, or any combination of [a], [b] and [c].

Embodiments and implementations of the invention will be apparent to those skilled in the art from a consideration of the specification or practice of the invention disclosed herein. It is intended that the specification and examples be considered as exemplary only, with the true scope of the invention being indicated by the following claims.

What is claimed is:

1. A system for adapting use of dialysis solution bags between modalities of dialysis treatment, comprising:
an adaptor assembly adapted for coupling a dialysis solution bag for a first modality of dialysis treatment to a disposable set configured for connection to a patient for a second modality of dialysis treatment, wherein fluid flow from the dialysis solution bag is controlled by the adaptor assembly for use with the second modality of dialysis treatment.

2. The system according to claim 1, wherein the first modality of dialysis treatment is automated peritoneal dialysis (APD) and the dialysis solution bag is an APD solution bag, and wherein the second modality of dialysis treatment is a manual exchange treatment and the disposable set is a manual exchange disposable set.

3. The system according to claim 2, wherein the adaptor assembly includes a manual exchange solution bag acting as a reservoir fill bag and is coupled to the APD solution bag via a connection adaptor device.

4. The system according to claim 2, wherein the adaptor assembly includes a flow limiter device that limits fluid flow to the manual exchange disposable set.

5. The system according to claim 4, wherein the flow limiter device includes an impeller that delivers a controlled amount of fluid to the manual exchange disposable set.

6. The system according to claim 4, wherein the flow limiter device includes a clamping mechanism that engages to stop fluid flow.

7. The system according to claim 6, wherein the clamping mechanism includes a spring-loaded clamping mechanism.

8. The system according to claim 4, wherein the flow limiter device includes an ultrasonic sensor that uses ultrasonic waves to determine fluid flow and that engages to limit fluid flow.

9. The system according to claim 8, wherein the ultrasonic sensor triggers a clamping mechanism to stop fluid flow.

10. The system according to claim 9, further comprising an ultrasonic piezo motor, wherein the ultrasonic sensor engages an ultrasonic piezo motor to trigger the clamping mechanism.

11. A method for adapting use of a dialysis solution bag between modalities of dialysis treatment, comprising:
providing a dialysis solution bag for a first modality of dialysis treatment;
coupling the dialysis solution bag to a disposable set for a second modality of dialysis treatment via an adaptor assembly;
controlling flow of fluid from the dialysis solution bag via the adaptor assembly in a manner suitable for use with the second modality of dialysis treatment.

12. The method according to claim 11, wherein the first modality of dialysis treatment is automated peritoneal dialysis (APD) and the dialysis solution bag is an APD solution bag, and wherein the second modality of dialysis treatment is a manual exchange treatment and the disposable set is a manual exchange disposable set.

13. The method according to claim 12, wherein the adaptor assembly includes a manual exchange solution bag acting as a reservoir fill bag and is coupled to the APD solution bag via a connection adaptor device.

14. The method according to claim 12, wherein the adaptor assembly includes a flow limiter device that limits fluid flow to the manual exchange disposable set.

15. The method according to claim 14, wherein the flow limiter device includes an impeller that delivers a controlled amount of fluid to the manual exchange disposable set.

16. The method according to claim 14, wherein the flow limiter device includes a clamping mechanism that engages to stop fluid flow.

17. The method according to claim 16, wherein the clamping mechanism includes a spring-loaded clamping mechanism.

18. The method according to claim 14, wherein the flow limiter device includes an ultrasonic sensor that uses ultrasonic waves to determine fluid flow and that triggers action to limit the fluid flow.

19. The method according to claim 18, wherein the ultrasonic sensor triggers a clamping mechanism to stop fluid flow.

20. The method according to claim 19, wherein the ultrasonic sensor engages an ultrasonic piezo motor to trigger the clamping mechanism.
